(19) United States
(12) Patent Application Publication
(10) Pub. No.: US 2007/0135876 A1
(43) Pub. Date: Jun. 14, 2007

(54) Acne and skin defect treatment via non-radiofrequency electrical current controlled power delivery device and methods

(76) Inventor: Paul Joseph Weber, Fort Lauderdale, FL (US)

Correspondence Address: Paul Joseph Weber, Suite 400, 5353 North Federal Highway, Fort Lauderdale, FL, 33308, US

(21) Appl. No.: 11/297,600

(22) Filed: Dec. 8, 2005

(51) Int. Cl. A61N 1/00

(52) U.S. Cl. 607/99

(57) ABSTRACT

A device is described that can be used to treat acne and certain skin defects quickly, accurately, and economically by the controlled application of NON-radiofrequency electrical energy. This economical hand-held device is to be used by patients alone or with under the consultation of a physician. Spot application of the tip by the patient for only seconds with or without a conductive gel or liquid adjunct allows electrical energy access below the surface of the skin without creating significant epidermal damage. This electron flow through the base of the lesion to be treated unique to this device; in the instant invention, the electron flow is diametric across the tip dimensions between the electroconductive tip elements, but taking the path of least resistance, that being through the depth of the acne lesion to be treated. The invention also pertains to attendant methods for enhancing the energetic effects of the electrical delivery device via concurrent external application of organic and inorganic, chemicals and materials.

### BACKGROUND OF THE INVENTION

1. Field of the Invention

The invention relates to acne and skin defect treating devices. More specifically, however, this application discusses the improvement of acne and skin defects via precise application of NON-radiofrequency electrical energy. The instant invention relates to an economical, hand-held device to treat acne lesions, more rapidly than currently possible. The device is to be used by patients alone or with the assistance of a physician. The non-invasive cosmetic device is placed in contact with the surface skin or just above the surface of the target skin (with or Without a conductive gel or liquid adjunct) with a tip geometry that allows electrical energy to spread like miniature lightning below the surface of the skin without creating significant epidermal damage. This electron flow through the base of the lesion to be treated unique to this device. In the instant invention, the electron flow is diametric across the tip dimensions between the electroconductive tip elements, but taking the path of least resistance, that being through the depth of the acne lesion to be treated. The invention can be used by medical personnel or the lay public in one or many spot treatments taking less than a second each to treat various areas and volumes of affected tissues. No anesthetic is necessary, yet recovery from the treatment is rapid; female patients may apply makeup to the treated areas to hide any residual pinkness. The invention may also include concomitant external application of organic and inorganic, chemicals and materials to enhance conductivity or medically assist acne or skin defect improvement. Unique and important side-benefits of using this device is also the reduction in milia, sinus wall three-dimensional characteristics, pore wall 3-D reduction as well as improvement in texture, wrinkling, undulations, folds or other defects in the surface tissues.

2. Description of Related Art

Animal and human skin is usually composed of at least 3 layers. These layers include the: outermost surface epidermis which contains pigment cells and pores, the dermis or leather layer, and the subdermis which is usually fat, fibrous tissue or muscle. The current target of most acne treatments is the epidermis and upper dermis wherein lie the follicles or pores (with or without hair) that may have retained debris become inflamed. Many medical regimens include the use benzoyl peroxide or topical antibiotics (erythromycin, tetracycline-derivatives, clindamycin, etc.) to treat acne. These topical or oral agents penetrate into and around the pore altering the local chemistry and cellular populations to reduce inflammation and bacterial populations. Acne surgery is a more precise mechanical acne treatment usually performed by an aesthetician, nurse or physician and includes techniques such as probing acne with sharp needles, scalpels or scissors. Disturbing acne lesions by “popping pimples” is a common technique used by younger patients and may be successful in reducing the time an acne lesion is prominent, however crude techniques such as this suffer from the disadvantage that pressure may rupture the acne inward into the skin's layers thus causing excess inflammation and even scarring. Chemical peeling agents such as acids and irritating microdermabrasion crystals have been known to improve acne as well as other skin defects. Sunlight and photodynamic therapy (light activated creams or pills) have been known to improve acne likely via modulating the skin's immune system and thus the inflammatory cell response. Other skin traumatizing devices such as lasers and phototherapy have been known to improve acne and skin defects to a certain extent, however such devices are usually expensive, bulky and administered by costly health care professionals. High electrical energy radiofrequency devices are occasionally used by doctors to lance the skin near acne lesions in a process similar to depilation; however, radiofrequency generators are of cumbersomely large size, dangerous for lay operation and very expensive. For example, U.S. Pat. No. 5,419,344 of Dewitt relates to a surgical method of razor bump electrolysis and surgical ‘scalpel’ shaving; this is a surgical procedure to be carried out by a health care professional, clearly not low cost or time effective on a daily basis for an average teenager. Another example is U.S. Pat. No. 4,846,179 of O'Connor describing a medical apparatus to treat folliculitis and skin infections using electrosurgery with a spark gap. Spark gaps result in high temperatures and great tissue damage and therefore excess tissue death and a likelihood of scarring. Depending upon how severe the underlying condition is, some scarring may be acceptable. However, in an average teenager, scarring is unacceptable as well as daily surgical procedures. Perhaps the underlying theme of most methods and treatments competing to solve acne is, at least temporary skin irritation and immune modulation. As was just discussed in the previous examples, the irritation can border on damage and surgery. All other therapies take time to treat acne, as opposed to the instant invention. Most of these treatments affect the epidermis; the epidermis is very delicate and prone to permanent problems if damaged. The current non-radiofrequency invention attempts to minimize interaction with the delicate epidermis.

Depilating (hair removing) equipment removes hair by having a medical professional pass an electroconductive needle deep into the hair pore while applying electrical energy often the radiofrequency type from a radiofrequency generator. However, this is impractical for personal, home use due to high cost and the danger of misuse wherein such levels of electrical energy can alter the epidermis causing scarring.

Recently, simple skin heating element systems like Zeno™ have been introduced (by Tyrell Inc., Houston, Tex., telephone 281-880-6541, U.S. Pat. Nos. 6,635,075 and 6,245,093) to allow the general public to treat their own acne via a hand held thermal energy device. Zeno's system suffers from needing to press the heating tip against the skin for over 1 to 2 minutes for EACH acne spot; thus, it could take over a half-hour to heat treat fifteen or more lesions, too long for rushing teenagers. Another system developed by Dermacare (Livermore, Calif.) heats the acne lesions with a similar device but takes less time; however, excess surface heating may result from too rapid a heat transfer possibly resulting in the adverse results of pigmentation changes, atrophy and even scarring. These devices are like placing miniature irons or heating elements externally on the surface of the skin; passing heat energy through the delicate epidermis to get to the lower portions of an acne lesion carries attendant risks.

When a disturbance occurs in the dermis such as trauma, fibroblasts are activated and not only produce new reparative strengthening collagen but contract, thus tightening and sealing healing tissue. Thermal damage to collagen may be brought about by hydrolysis of cross-linked collagen molecules and reformation of hydrogen bonds resulting in loss of portions or all of the characteristic collagen triple-helix. Nonetheless, a controlled and uniform trauma to the dermal layer can be medically beneficial and visually desirable as can be seen in cases of previously sun-damaged women's faces following a deep chemical peel.

Currently, a need exists for a personal hand-held rapid acne treatment device with the following assets: 1) rapid treatment ability, 2) extremely low risk, minimal epidermal irritation, 3) ease of home use, 4) low cost, 5) low pain during application, 6) safety which cannot be achieved by radiofrequency, 7) direct subsurface tissue energy application capability to alter inflamed pores as well as to induce or stimulate fibroblasts/collagen resulting in skin tightening and strengthening thus completely bypassing the ultra-sensitive and fragile epidermis and thus avoiding visible surface scaring and pigment loss/excess.

Applicant meets the needs. Currently no device or method in the medical literature addresses all of these concerns simultaneously. After the insertion of simple tumescent anesthesia, a human facial procedure is estimated to take only 15 minutes to perform in experienced hands, including stitching.

The paper-thin layer of the skin that gives all humans their pigmentary color and texture is the epidermis. Unfortunately, virtually every skin rejuvenation system that has existed until now (with the exception of injectable skin filling compounds) and even traditional face-lifting surgery (when cutting through the skin around the ear is considered) must pass through the epidermis to attempt to reach and treat the dermis. Damage to the epidermis and its component structures may results in undesirable colorations or color losses to the skin as is seen in scarring. The prime consideration over the last decade for scientists and engineers regarding skin rejuvenation procedures is how to spare damage to the thin but critical epidermis and adjoining upper dermal layer.

Monopolar radiofrequency electrosurgical instruments possess a single active electrode at the tip of an electrosurgical probe. Low voltage applied to the active electrode in contact with the target tissue moves electrical current through the tissue and the patient to a dispersive grounding plate or an indifferent electrode. Voltage differences between the active electrode and the target issue cause an electrical arc to form across the physical gap between the electrode and tissue. At the point of arc contact with tissue, rapid tissue heating occurs due to high current density between the electrode and tissue. Current density causes cellular fluids to vaporize into steam yielding a cutting effect. Monopolar electrosurgery methods generally direct electric current along a defined path from the active instrument electrode through the patient's body into the return or grounding electrode. Small diameter electrodes increase electrical field intensity in the locality. Bipolar configurations more easily control the flow of current around the active region of a treatment device which reduces thermal injury and thus minimizes tissue necrosis and collateral tissue damage while reducing conduction of current through the patient.

Eggers in U.S. Pat. No. 5,871,469 and related patents differs from applicant who does not use radiofreqency energy. Eggers teaches a radiofrequency electrosurgical device that requires an ionic fluid to create conduction between very minute arrayed electrodes and relies on an ionic fluid source from within the instrument to function optimally; this is impractical for home use. Eggers teaches bipolar energy flows principally between pairs or groups of minute electrodes arranged in various arrays depending upon the embodiment chosen. The geometry of Eggers would fail to get through to the bottom of an acne lesion as well as the instant invention. To quote Eggers in U.S. Pat. No. 5,871,469 column 4 line 49: “The electric field vaporizes the electrically conductive liquid into a thin layer over at least a portion of the active electrode surface and then ionizes the vapor layer . . . ”. Eggers teaches vaporizing a thin layer of an optimizing conducting fluid; additional application of a conducting fluid is not a necessity for applicant. Eggers furthermore reveals in column 11: “The depth of necrosis (tissue death, lethal alteration) will typically be between 0 to 400 microns and usually 10 to 200 microns (=0.2 mm).” This is clearly too much damage at the delicate surface epidermis while not penetrating to the 2 mm depth of a typical acne lesion; thus Eggers penetrates only 10% of the way or 0.2/2 through the average acne pimple. Eggers' U.S. Pat. No. 5,871,469 external skin resurfacing (Visage®) requires an external ionic fluid drip and has been in clinics and is known not to remove much more than very fine wrinkles without epidermal pigment changes or scarring. Only the thinnest wrinkles can be reduced by Visage®.

Eggers' U.S. Pat. Nos. 6,740,079 and 6,719,754 and 6,659,106 and 6,632,220 and 6,632,193 and 6,623,454 and 6,595,990 and 6,557,559 and 6,557,261 and 6,514,248 and 6,482,201 and 6,461,354 and 6,461,350 are virtually all bipolar radiofrequency in nature and require a fluid delivery element that may be located on the probes or part of a separate instrument. Alternatively, an electrically conducting gel or spray may be applied to the target tissue. This art would not be safe, practical for rapid home use in the patient population.

Thermage, Inc. of Hayward, Calif. recently introduced to the market its radiofrequency based tissue contraction product of an externally applied electrosurgical template activated while touching the outer. Energy passes through the epidermis thus passing energy through the upper skin with the intention of electrically altering collagen to achieve remodeling; damage to the epidermis is reduced some by externally spraying a cryogen (cooling gas) of about −40° C. on the targeted zone's epidermis at the time of the electrical impulse. Unfortunately, the amount of tissue contraction Thermage, Inc. can prove in the medical literature borders upon statistical insignificance (to quote several prominent cosmetic surgeons) and is far less than 5%. U.S. Pat. No. 6,413,255B1 of Stern relates to Thermage's device and is an externally applied “tissue interface surface . . . and has a variable resistance portion.” '255B1 teaches a linear array of externally applied bi-polar radiofrequency electrodes; an externally applied monopolar embodiment using return electrodes is also illustrated. Base claims in '255B1 regarding the radiofrequency electrosurgical delivery device indicate contact with the skin's external, outer surface. Knowlton: U.S. Pat. Nos. 6,470,216 and 6,461,378 and 6,453,202 and 6,438,424 and 6,430,446 and 6,425,912 and 6,405,090 and 6,387,380 and 6,381,498 and 6,381,497 and 6,337,855 and 6,377,854 and 6,350,276 and 6,311,090 and 6,241,753 and 5,948,011 and 5,919,219 and 5,871,524 and 5,755,753 are Thermage, Inc. licensed. Knowlton mentions in '498B1 “Instead, . . . a partial denaturization of the collagen permitting it to become tightened.” Nonetheless, the ultrapowerful radiofrequency can cause permanent epidermal and dermal scarring.

### SUMMARY OF THE INVENTION

It is an object of the present invention to provide a device and method that can be used by the lay public, to provide quick, accurate and inexpensive acne and facial defect care in the privacy of their own home.

It is another object of the invention to reduce complications associated with other acne and skin defect treatments. The preferred embodiment of the invention is a hand-held, self-contained non-radiofrequency electrical power generation and delivery device. A variety of tip shapes and accessorize may be used to personalize the device.

Topical applications of water bases electroconductive or ionic materials may be used to aid in efficacy. Said applications may also contain other agents currently know to improve acne such as antibiotics, benzoyl peroxide, retinoids, etc.

The present invention can be used to improve the efficacy and safety of acne treatment. The forgoing and other objects, features, and advantages of the present invention will become apparent from the following description and accompanying drawings.

### BRIEF DESCRIPTION OF THE DRAWINGS

FIG. 1 shows side external view of the handle and tip of a typical embodiment of the invention in contact with an acne lesion.

FIG. 2 shows top view of the internal workings of a typical embodiment of the invention contained with the handle and tip.

FIG. 3 shows various tip designs that may be placed into the receptacle/socket.

FIG. 4 shows block diagram or a logic flow sheet for function of the invention.

### DETAILED DESCRIPTION OF THE INVENTION

The present invention is a device that can be used quickly and accurately by patients on their acne and facial defects. The hand-held device is comprised of an internal battery, time and power selection via potentiometer, logic control circuit for duration and delivery of power, power generation circuit comprised of resistive/capacitive networks, switching transistors, optional enunciators or light emitting diodes, and conductive treatment tips, all encased in a non-conductive self-contained unit. Digital interfaces or dials can be used to alter the duration, frequency and intensity or current flow for optimal tissue treatment.

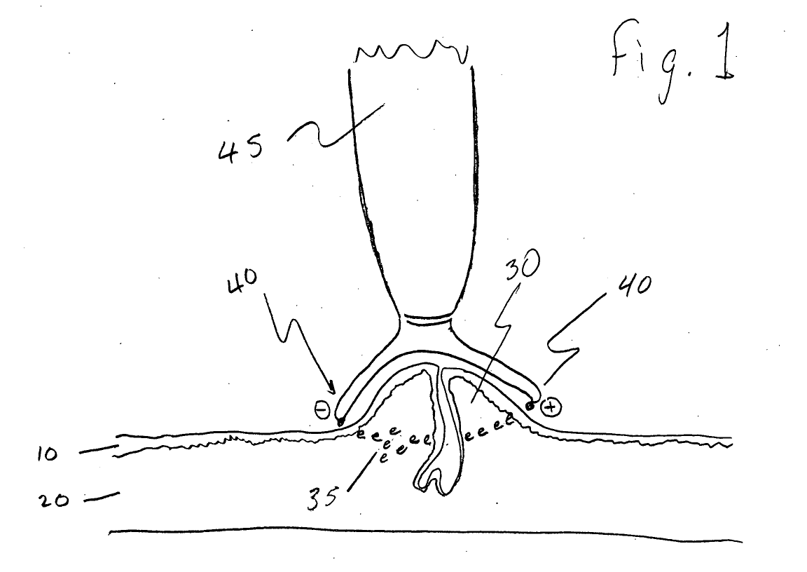

FIG. 1 shows side external view of the handle and tip of the electrical current embodiment of the invention in contact with an acne lesion. The epidermis 10 overlying dermis 20, containing an acne lesion is located about a hair follicle 30. A stream of (e) electrons 35 flows like lightning from the terminals located on conductive tip 40 through the depth base tissue of acne lesion derived from hair follicle 30. This electron flow through the base of the lesion to be treated unique to this device. In FIG. 1, the non-linear path depicted for the stream of “e” is meant to indicate some deviations like a lightning bolt in the sky are possible but that the main flow of electrons is through the depth of the lesion. The electron flow is diametric across the tip dimensions between the electroconductive tip elements, but taking the path of least resistance, which should be close to the straightest, that being through the depth of the acne lesion to be treated. The electroconductive tip elements 40, are controlled and supplied with power via handle 45 which may or may not be at a greater distance from tip if a shaft is interposed between the tip and handle 45.

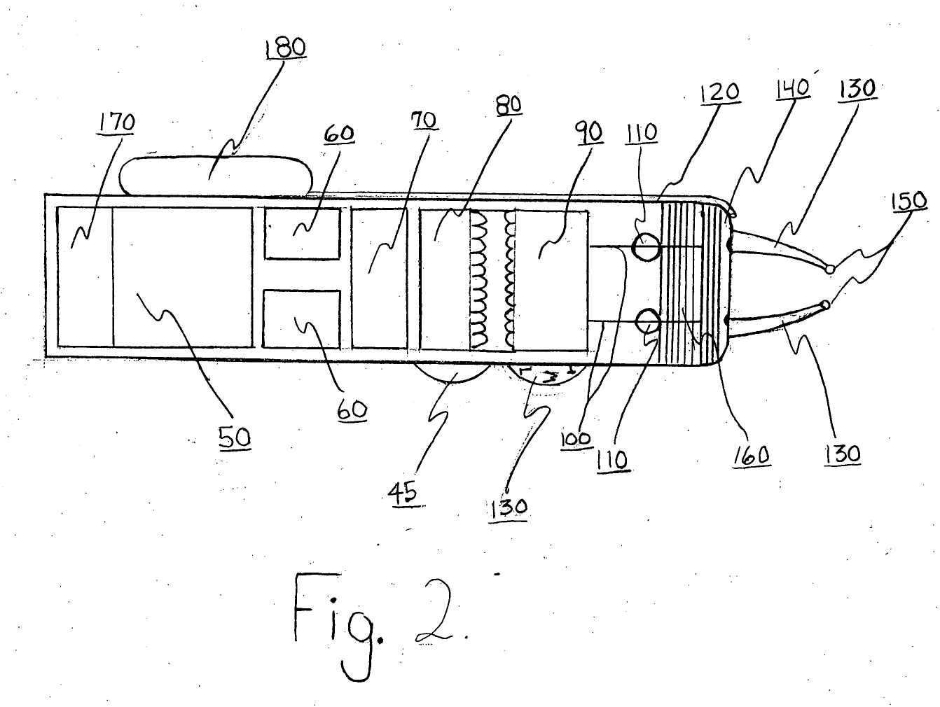

FIG. 2 shows top view of the internal workings of a typical embodiment of the invention contained within the handle and tip. A battery 50 provides, by conductive elements, initial power to logic circuits 60 (Texas Instruments, Dallas, Tex.) which are connected to capacitor 70 (Tyco International, Portsmouth, N.H.). The capacitive network 70 is further connected to the resistive/capacitive network 70 which is in turn connected to the first half of the isolation transformer 80 which approximates the second half 90 of the isolation transformer which is connected by two or more conductive elements numbered 100 to light emitting diode indicators 110. Conductive elements 100 are further connected to socket 140 to which are attached accessory contact leads/tips/plate 130 to which are attached the accessory tissue contact points 150. An optional heat sink 160 made of dense materials or metals such as steel or copper or aluminum can allow the device to be placed into the refrigerator or freezer prior to treatment to reduce pain upon treatment by cooling the skin on contact; the heat sink may or may not be a part of the socket or accessory tissue contact plate or cup. This effect is much like using an ice cube to cool a wound when someone is injured, just before instead. An optional piezoelectric generator 170 (10 to 1,000,000 hertz) may be found at any location within the device, but preferably at the farthest point away from the treatment tip, to generate further tip transfer energy into the target ache lesion; however, in this case the energy will be vibrational. An optional cooling gas or liquid reservoir 180 may be found at any location within or on the outside of the device to shoot cold gas or liquid into the treatment area. Optional cooling liquids or gels may contain electroconductive ions or materials to enhance electrical energy transmission into the skin. Optional cooling gasses include, but are not limited to, carbon dioxide, chlorofluorocarbons, halogenated hydrocarbons, compressed air etc. which would make use of any property including boiling point and Boyle's law to cool the skin. Also optionally, the cold side of a Peltier thermoelectric cooler may be located near the tip attachment so that skin nearing or in the treatment zone in contact with cold side of a Peltier thermoelectric cooler may feel less pain.

Battery 50 types may include rechargeable nickel-cadmium, lithium, nickel metal hydride, or disposable alkaline or standard lead acid. Batteries may range from 6-12 Volts and is about 500 milliamp-hours. Leaving the battery, standard battery wiring or board mount connectors provide initial power usually ranging from 1-12 Volts to the logic circuits 60 (Texas Instruments, Dallas, Tex.). The logic circuits control all functions, including but not limited, to “watch dog” circuitry, output control, feedback, impedance measurement, time/timing, operating parameters, and over-current protection. The logic control circuits are connected via runs on circuitboard to the capacitor and to virtually all other functions. The capacitive network 70 stores electrical energy to increase the voltage (to levels of 10 to 25 volts) for the proper treatment. A step up isolation transformer further increases the voltage to reach levels of 0-300 Volts with a current of 0-2,750 milliamps (mA). The most preferable voltage ranges from 20-60 Volts with a preferable current range of 100-500 mA. A desirable contact tip resistance ranges from 500-1500 Ohms whereas non-contact resistances could be infinite. The second half of the isolation transformer steps up voltage to the output or tips of the device. The transformer is customarily made of copper wound coil. Standard wire or circuitboard connects light emitting diode indicators (preferably red for stop and green for go). A heat sink, preferably aluminum, is a passive device that draws heat away from the tip if any is produced. A partially or fully conductive socket locks in the accessory treatment tips by friction or locking capture mechanism. The optional piezoelectric generator (10 to 1,000,000 hertz) is made of ceramic sensitive to oscillating current and ranges from 100 millivolts to 2 Volts.

The handpiece may be made of metal or plastic or other material with a completely occupied or hollow interior that can contain insulated wires, electrical conductors, fluid/gas conduits, or insulation. Plastics, such as Teflon® may act as insulation about wire or electrically conductive elements. A handpiece made of metals or alloys must contain sufficient insulating materials within to prevent unwanted discharge or conduction between internal elements and the skin or tip. The handpiece may alternatively be made partially or completely of concentrically laminated or annealed-in wafer layers of materials that may include plastics, silicon, glass, glass/ceramics, ceramics carbon, graphite, graphite-fiberglass composites. Impedance values may be tracked on a display screen or directly linked to a microprocessor capable of signaling control electronics to alter the energy delivered to the tip when preset values are approached or exceeded. Although options exist for self-contained cooling of the apparatus tip External cooling devices may be applied to the facial skin before, during or after treatment, for example, ice cool water soaked towels or ice cold water circulated through a externally conforming bag to enhance the reverse thermal gradient.

The instant invention is basically a hand-held acne and facial defect treatment apparatus, comprising a tip and a handpiece; and electrical energy generation means for producing non-radiofrequency pulses, and a means to regulate the energy type delivered to said tip and a delivery means for transmitting said electrical energy to a geometrically styled tip wherein electron flow is diametric across or between the tip dimensions comprised of a plurality of electroconductive tip elements, thus electron flow is through the depth of the target lesion.

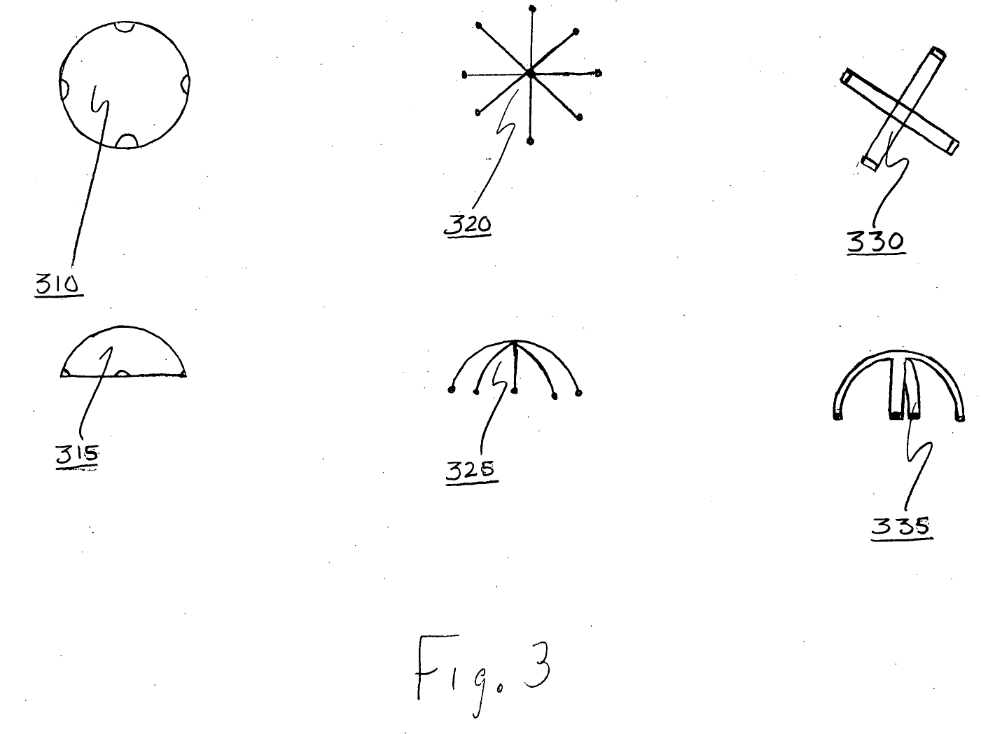

FIG. 3 shows various tip designs that may be placed into the receptacle/socket. However, the tip protrusion shapes may take on a wide variety of geometric shapes. 310 is a top view (whereas 315 is a side-view) of the cup-shaped, semi-spheroidal tip of 3-4 mm in diameter best shaped to fit over a typical acne lesion. Electroconductive electrodes are spaced around the circumference in multiples of two because of bipolarity. The remainder of the cup is nonconductive and made from materials which include those mentioned in general for tip construction in this patent. Unfortunately, clear zirconium would need to comprise the cup-shaped treatment tip accessory to allow visualization during treatment. 320 is a top view (whereas 325 is a side-view) of the “spider” electrode pattern made of alternating active and dispersive electrodes around a circumference but all open to “air” and not retained by an insulating cup. This pattern allows clear visualization of the treatment zone. 330 is a top view (whereas 335 is a side-view) of the “cross” pattern electrode made of a nonconductive arms with conductive elements within the arms leading to the exposed electroconductive elements on the tips of the arms. The tip again can be of a multitude of geometric shapes and conformations as well as the spacing of the array of electroconductive tip elements scattered about the tip geometry. Tip geometry may be concave, convex and planar. The planar conformation may even allow for proper electron flow through the base of lesions under certain circumstances. Tip and surrounding elements may made of a variety of electroconductive and non-electroconductive elements. For example, stainless steel, aluminum, copper, gold, silver, platinum, titanium may comprise the electroconductive elements. Nonconductive elements may include the likes of alumina, zirconia, glasses or ceramics. A favored ceramic for tip construction is Forsterite of 2.9 g/cm3 density, flexural strength of 1500/kg/sqcm, temperature expansion coefficient (83+/−5)10E-7, composition: Al₂O₃ 0.8%, SiO₂ 41.7%, MgO 51.5%, BaO 6%. Another favored ceramic for tip construction is BK 94-1 (Russian Index), flexural strength of 3200/kg/sqcm, composition: Al₂O₃ 94.4%, SiO₂ 2.8%, MnO₂ 2.3%, Cr₂O₃ 0.5%. it is the apex of repetitive spokewheel passages. Electrically conductive element can be in the shape of a plate or plane or wire and made of any metal or alloy that does not melt under operating conditions or give off toxic residua; optimal materials may include but are not limited to steel, nickel, alloys, palladium, gold, tungsten, copper, and platinum. These metals can become oxidized thus impeding electrical flow and function. Calculated oxidation of the electrically conductive elements may be used to plan obsolescence so that one embodiment of the device may be a low cost, disposable, or restricted-use device. However, other embodiments intended for multiple use require the tip's electrically conductive tissue lysing elements be protected or coated with materials that include but are not limited to Silverglide™ non-stick surgical coating, platinum, palladium, gold and rhodium. Varying the amount of protective coating allows for embodiments of varying potential for obsolescence capable of either prolonging or shortening instrument life. The electrically conductive element portions of the tip may arise from a plane or plate of varying shapes derived from the aforementioned materials by methods known in the manufacturing art, including but not limited to cutting, stamping pouring, molding, filing and sanding. This electrically conductive elements may be an insert attached to a conductive element in the shaft or continuous with a formed conductive element coursing all or part of the shaft. Alternatively, electrical current can cause an effect at a distance without direct contact the to electrically conductive element which may be recessed into the tip or flush with it. Adjustable, locations of the electrically conductive elements with respect to the treatment tip may be achieved by diminutive screws or ratchets. Other tertiary shapes may include but are not limited to straight bristle shaped, bent bristle shaped, bristle shaped atop a cone, bristle shaped distally atop spring shape proximally, and bristle shapes with further branched bifurcation or “frizzies”.

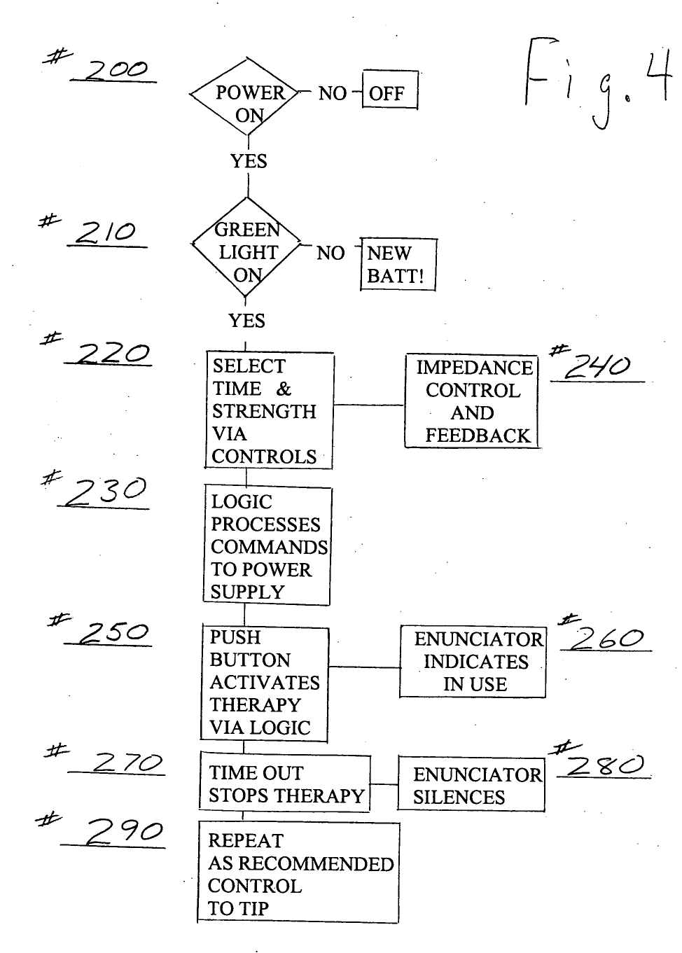

FIG. 4 is a logic flow sheet. 200 describes the “on-off” switch. 210 represents a light emitting diode indicator allowing patients to know that the device has been turned on and is ready for use. 220 relates to the patient selection of timing of current burst(s) and strength (energy). 230 is the logic processes command power supply which initiates the prior selection of time duration and strength of energy selection. 240 is the impedance feedback control logic chip function that regulates the electrical energy output as a function pretreatment skin conductivity or changes in skin conductivity brought about by just previous treatment(s) (for example, treatments varying from 1 millisecond to hours before). 250 represents the push button activation itself to deliver energy to the tip. 260 shows an enunciator (sound or visible) which is a further safety indication of use. 270 is the “time-out” function that stops therapy and is basically a circuit cut-off. 280 represents the enunciator silences function which alerts the user that therapy is properly complete. 290 discussed the request to the patient or the clinician overseeing the patient to repeat the therapy as necessary.

The foregoing description of preferred embodiments and methods of use of the invention are presented for purposes of illustration and description and are not intended to be exhaustive or to limit the invention to the precise form disclosed. Many modifications and variations are possible in light of the above teaching. The embodiments were chosen and described to best explain the principles of the invention and its practical application to thereby enable others skilled in the art to best use the invention in various embodiments and with various modifications suited to the particular use contemplated.

We claim:

1. A hand-held acne and facial defect treatment apparatus, comprising:
a tip and a handpiece; and
electrical energy generation means for producing non-radiofrequency pulses, and a means to regulate the energy type delivered to said tip and a delivery means for transmitting said electrical energy to a geometrically styled tip wherein electron flow is diametric across or between the tip dimensions comprised of a plurality of electroconductive tip elements, thus electron flow is through the depth of the target lesion.

2. A hand-held acne and facial defect treatment apparatus, comprising:
a tip and a handpiece; and
electrical energy generation means for producing non-radiofrequency pulses and delivery means for transmitting said electrical energy to a geometrically styled tip that approximates the dome shape of an acne lesion with a plurality of electroconductive elements and a means to regulate the energy type delivered to said tip.

3. A hand-held acne and facial defect treatment apparatus, comprising:
a tip and a handpiece; and
electrical energy generation means for producing non-radiofrequency pulses and delivery means for transmitting said electrical energy to a geometrically styled tip that approximates the dome shape of an acne lesion with a plurality of electroconductive elements and a means to regulate the energy type delivered to said tip; and a cooling means to reduce pain.

4. The apparatus of claim 1, wherein said. means for producing non-radiofrequency electrical energy pulses are chosen from a group at least consisting of a battery, logic circuits, capacitor, resistive/capacitive network, and isolation transformer.

5. The apparatus of claim 1, wherein said means for transmitting non-radiofrequency electrical energy pulses to the treatment tip are chosen from a group at least consisting of conductive elements, connecting socket, attached accessory contact leads/tips/plate with tissue contact points.

6. The apparatus of claim 1, wherein said means to regulate the energy characteristics appearing at the tip is chosen from a group at least consisting of the following logic circuits, electrical impedance detector, and electrical impedance feedback loop.

7. The apparatus of claim 1, further comprising a means connected to said tip for detecting the electrical impedance of the treated target tissue.

8. The apparatus of claim 1, wherein nonconductive portions of the tip are comprised of material selected from a group consisting of plastics, plastics including Teflon®, silicon, carbon, graphite, graphite-fiberglass composites, porcelain, epoxy, ceramic, glass-ceramics.

9. The apparatus of claim 1, wherein conductive portions of the tip are comprised of material selected from a group consisting of conductive metals, iron, steel, alloys, platinum, palladium, nickel, aluminum, titanium, gold, silver, and copper.

10. The apparatus of claim 1, wherein said tip conductive electrodes are seated in relation to the remainder of the tip in a location selected from a group consisting of protruding, flush with, recessed, concave, convex, and planar.

11. The apparatus of claim 1, further comprising means for controlling the character of the electrical energy available at the treatment tip as chosen from the group consisting of frequency, duration and intensity.

12. The apparatus of claim 1, wherein planned obsolescence is increased or decreased to said electrical energy delivery tip by the amount of cover for electrodes with an oxidation reducing material.

13. The apparatus of claim 10, wherein said oxidation reducing material is selected from the group of Silverglide®, Silverglide®-like coatings, alloys, gold, platinum, rhodium, and palladium.

14. The apparatus of claim 1, wherein tip is comprised of material that is electrically conductive, and tip is not insulated in at least one portion, and is not insulated at least one immediate point of contact with a means for providing electrical energy, and elsewhere tip is completely insulated.

15. The apparatus of claim 1, further comprising a least one impedance sensor that senses skin conditions at the tip, wherein said sensor sends a signal to control means, and wherein said control means controls the delivery of said energy to said distal end to modulate said inductance.

16. The apparatus of claim 1, wherein said electrically energized tip accessory design is of a physical characteristic chosen from the group of: geometric, cup, bowl-shaped, flat, scalloped, saw-toothed, flat, oval, circular, square, rectangular, triangular, trapezoidal, linear, geometric.

17. The apparatus of claim 1, wherein said tip electrodes are bipolar electrodes.

18. The apparatus of claim 3, wherein said means for providing cooling to the target tissue is chosen from the following group: cold side of a Peltier thermoelectric cooler, cold gas outlet, and heat sink.

19. The apparatus of claim 1, additionally including an optional ultrasonic transducer piezoelectric located within the handle and thus may impart ultrasonic energy to the device and tip.

20. A method for treating targeted tissue comprising:
delivering non-radiofrequency energy to target tissue via electroconductive materials at a treatment tip in combination with other acne treatments chosen from the group of: antibiotics, retinoids, chemical peeling agents, benzoyl peroxide.

21. A method for treating targeted tissue comprising:
delivering non-radiofrequency energy to target tissue via electroconductive materials at a treatment tip in combination with the application of electroconductivity enhancing materials chosen from the group consisting of ionic liquids, gels and solids.

22. A method for treating targeted tissue comprising:
delivering non-radiofrequency energy to target tissue via electroconductive materials at a treatment tip in combination with skin cooling methods chosen from the group of: cool gas emission, cool liquid emission, cool gel emission, cool side of Peltier cooler, and refrigerated heat sink.
